(19) United States
(12) Patent Application Publication
(10) Pub. No.: US 2025/0352940 A1
(43) Pub. Date: Nov. 20, 2025

(54) Soft Sensors for Estimating Operating Parameters in Reactive Absorption Units

(71) Applicant: Saudi Arabian Oil Company, Dhahran (SA)

(72) Inventors: Abdullah Al Ghazal, Dhahran (SA); Zahra Alshulah, Qatif (SA); Yufeng He, Dhahran (SA)

(21) Appl. No.: 18/664,707

(22) Filed: May 15, 2024

(51) Int. Cl. B01D 53/14; B01D 53/18; C10L 3/10; G16C 20/10; G16C 20/70

(52) U.S. Cl. CPC B01D 53/1412; B01D 53/1462; B01D 53/18; C10L 3/103; C10L 3/104; G16C 20/10; G16C 20/70; B01D 2252/204; B01D 2257/304; B01D 2257/504; C10L 2290/541

(57) ABSTRACT

A system and method for estimating a parameter for a reactive absorbance unit are provided. An exemplary method includes creating a kinetic model of an absorbance process, setting a range for each of a plurality of input parameters, based, at least in part, on operational data measured from the reactive absorbance unit. A sampling technique is used to generate a plurality of input vectors in the range of each of the plurality of input parameters. A plurality of output vectors is generated from the plurality of input vectors. A predictive model is trained with the plurality of output vectors and the plurality of input vectors. The parameter is estimated from the predictive model. The parameter is used in a control model for the reactive absorbance unit.

### TECHNICAL FIELD

This disclosure relates to methods for estimating operating parameters in reactive absorption units.

### BACKGROUND

Reactive absorption units, particularly amine absorption, are a common technology used in chemical engineering and the oil and gas industry for the removal of acidic gases, such as carbon dioxide (CO₂) and hydrogen sulfide (H₂S) from gas streams. The process begins by introducing the gas stream containing the acidic components into an absorption column. This gas stream typically comes from natural gas processing or industrial processes. An aqueous (typically amine) solution is circulated within the column and acts as an absorbent. The amine solution absorbs the acidic gases from the gas stream, effectively removing them. This results in an increase in the temperature of the amine.

The treated gas, now largely free of reactive components, exits the top of the column. The aqueous solution, now loaded with the absorbed gases, is sent to a regeneration unit. In this unit, heat is applied to release the captured gases, regenerating the amine solution for reuse. This heat driven process is called stripping or desorption. The stripped gases, which contain concentrated acidic components are processed further for disposal or other applications. The regenerated amine solution is then recirculated back to the absorption column to continue the cycle.

### SUMMARY

An embodiment described herein provides a method for estimating a parameter for a reactive absorbance unit. The method includes creating a kinetic model of an absorbance process, setting a range for each of a plurality of input parameters, based, at least in part, on operational data measured from the reactive absorbance unit. A sampling technique is used to generate a plurality of input vectors in the range of each of the plurality of input parameters. A plurality of output vectors is generated from the plurality of input vectors. A predictive model is trained with the plurality of output vectors and the plurality of input vectors. The parameter is estimated from the predictive model. The parameter is used in a control model for the reactive absorbance unit.

Another embodiment described herein provides a reactive absorbance unit. The reactive absorbance units includes a contactor. A feed gas line is coupled to the contactor proximate to the bottom of the contactor, wherein the feed gas line includes a sour gas feed. A sweetened gas line from the contactor is coupled proximate to a top of the contactor, wherein the sweetened gas line includes a sweetened gas. A lean amine line to the contactor is coupled proximate to the top of the contactor, wherein the lean amine line includes a lean amine solvent. A rich amine line from the contactor is coupled proximate to a bottom of the contactor, wherein the rich amine line includes an amine solvent with absorbed acid gases. The reactive absorption's unit includes a stripper, wherein the rich amine line is coupled to the stripper, and wherein the lean amine line exits the stripper after removal of acid gases. The reactive absorbance unit further includes a control system. The control system includes a processor and a data store. The data store includes instructions configured to direct the processor to obtain a data set for reactive absorbance unit, reconcile data imbalances in the data set, generate a plurality of input parameters for a kinetic model, run the plurality of input parameters in the kinetic model, and to generate a plurality of output parameters from the kinetic model. The data store also includes instructions configured to direct the processor to create a machine learning model relating input parameters to output parameters, and run new input parameters in the machine learning model to estimate an unmeasured parameter, creating an estimated parameter. The data store also includes instructions configured to direct the processor to perform a control calculation using the estimated parameter.

### BRIEF DESCRIPTION OF DRAWINGS

FIG. 1 is a drawing of a reactive absorption unit, showing a contactor and a stripper.

FIG. 2 is a drawing of a contactor used to absorb acid gases in a reactive absorption unit.

FIG. 3 is a process flow diagram of a method for using a model to optimize operations of a reactive absorption unit.

FIG. 4 is a schematic drawing of a kinetic model used to predict missing data from measured data.

FIG. 5 is a drawing of a one-dimensional convolutional neural network (1D CNN) that can be used to control the reactive absorption unit.

FIG. 6 is a block diagram of a system that uses a controller for optimizing operation of reactive absorption unit.

FIG. 7 is an example of an output screen that is used to control the reactive absorption unit, either through an open loop control or through a closed loop control.

### DETAILED DESCRIPTION

Embodiments described herein provide a method for optimizing the operation of reactive absorption units, such as acid gas strippers. The method uses a novel optimization technique to allow the operation of these units closer to their operational limits, therefore maximizing throughput or minimizing energy consumption, while protecting the units' metallurgy and preventing gas breakthrough.

The method uses machine learning techniques to capture spatial and sequential dependency of input parameters. For example, in some embodiments, 1-D convolutional neural networks (1D-CNN) are used, as they are effective in modeling parameters that have spatial dependency. This allows the modeling model of a temperature profile in a column that includes a number of trays. By comparison, modeling the temperature of each tray independently is likely to provide poor results. In addition to the 1D-CNN, other types of models, for example, based on polynomial regression are used other properties as described herein. Other types of models may be used in the process, including, for example, standard convolutional neural networks (ANN), in addition to or in place of the models mentioned above.

Further, if parameters are not measured, or missing due to instrument failures, the techniques allow for the estimation of missing values. For example, a kinetic model can be developed to model the reactive absorption process and predict output values over a wide range of input values. The predicted output values can then be used to train various types of models, such as regression models and machine learning models. The trained models can then be used to predict the missing values from current input values.

The proposed framework allows for the control of the reactive absorption units in either a closed loop or an open-loop system. As used herein, a closed loop control system interacts directly with the plant control system, and an open loop system provides a real-time advisory to an operator. In some embodiments, the model resides on a computer within the plant control network. It runs on a set frequency, for example, hourly, and sends operational adjustments directly to the plant's multi-variable control system or the regulatory control or displays recommended values on a monitor for the plant control system. In some embodiments, the model resides in a corporate IT network, in addition to or instead of the plant control network.

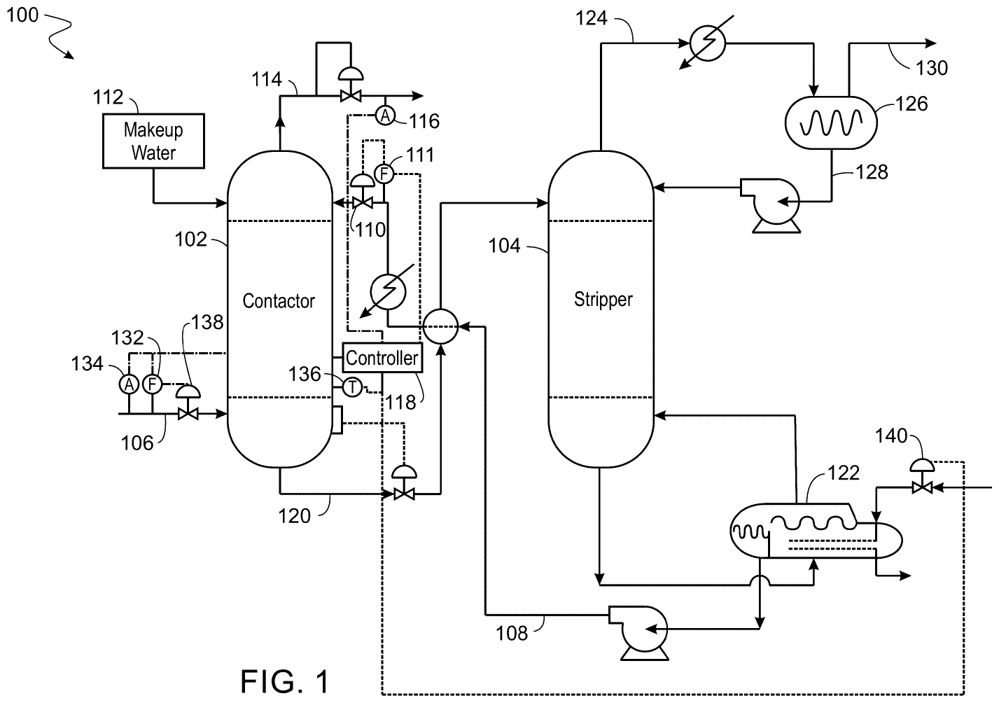

FIG. 1 is a drawing of a reactive absorption unit 100, showing a contactor 102 and a stripper column 104. A sour gas feed 106 is fed to the contactor 102 for sweetening. As used herein, sweetening is the lowering of the concentration of acid gases, such as H₂S and CO₂, for example, to within specification limits for sales gas. The H₂S in sour gas fed is often about 2-3 mol. % and is reduced to below 4 ppm in product sweet gas. For the acid gas, it is usually 10-14 mol. % in the feed. For example, this may be less than 500 ppm acid gas, less than 100 ppm, or less than 50 ppm. In the contactor 102, the sour gas feed 106 rises up through the contactor 102 through a series of trays. In some embodiments, the lowest tray is numbered 1 while the highest tray is numbered 30, although the number of trays may vary, for example, based on the expected concentration of acid gases in the sour gas feed 106.

In the contactor 102, a stream of lean solvent 108 is introduced near the top of the contactor 102, for example, above tray 30, through a flow control valve 110. The flow control valve 110 has an associated flow sensor 111 with control circuits to implement a proportional-integral-derivative (PID) control loop for controlling the flow of the lean solvent 108 into the contactor 102.

The solvent is a solution of the amine with water. In various embodiments, the solvent is a mixture of methyldiethanolamine (MDEA) and piperazine. In other embodiments, it is MDEA, diglycolamine (DGA), or mixtures thereof. As water is lost through the process, more water may be added from a makeup water system 112.

A sweetened gas stream 114 exits the contactor 102 from the top of the vessel. The sweetened gas stream 114 typically goes through a dehydrator as it is saturated with water. From there it goes sales gas compressor or to a natural gas liquids (NGL) fractionation to recover heavier hydrocarbons. An acid gas analyzer 116 may be used to determine the concentration of acid gases in the sweetened gas stream 114. This value is provided to a controller 118 to assist in determining a set point for the flow rate of the lean solvent 108 added to the contactor 102 through the flow control valve 110, for example, using the 1D-CNN and other models. In some embodiments, the measurements from the acid gas analyzer 116 are simulated.

After the lean solvent 108 passes through the contactor 102 and adsorption acid gases it exits the contactor 102 as a rich solvent 120. The rich solvent 120 is then flow to a stripper column 104 for removal of the acid gases and regeneration of the lean solvent 108. The stripper column 104 is configured as a standard separation column with a reboiler 122 providing heat to the solvent in the bottoms of the stripper column 104. From the top of the stripper column 104, a vapor stream 124 is cooled and passed to a reflux drum 126 for separation of liquids 128, such as the solvent, which are carried overhead. The liquids 128 are pumped back to the stripper column 104 as a reflux stream. The vapor 130 from the reflux drum 126 is an acid gas stream that may be provided to a sulfur recovery plant or disposed of.

The controller 118 uses a number of inputs to process the model and maximize the rate of the sour gas feed 106 or minimize the energy consumption. For example, the model can be used to calculate a set point for the flow rate of the lean solvent 108 fed to the contactor 102 through the flow control valve 110. The unit feed rate is determined by a flow sensor 132 on the sour gas feed 106 to the contactor 102. The feed gas composition is determined by an acid gas analyzer 134 on the sour gas feed 106 or is calculated as a virtual measurement through a model. Further inputs to the controller 118 can include the strength of the solvent solution and the solvent circulation rate, including, for example, the amine circulation rate, the amine concentration, and the concentrations of other materials, such as triethylene glycol (TEG) or piperazine (pz), among others.

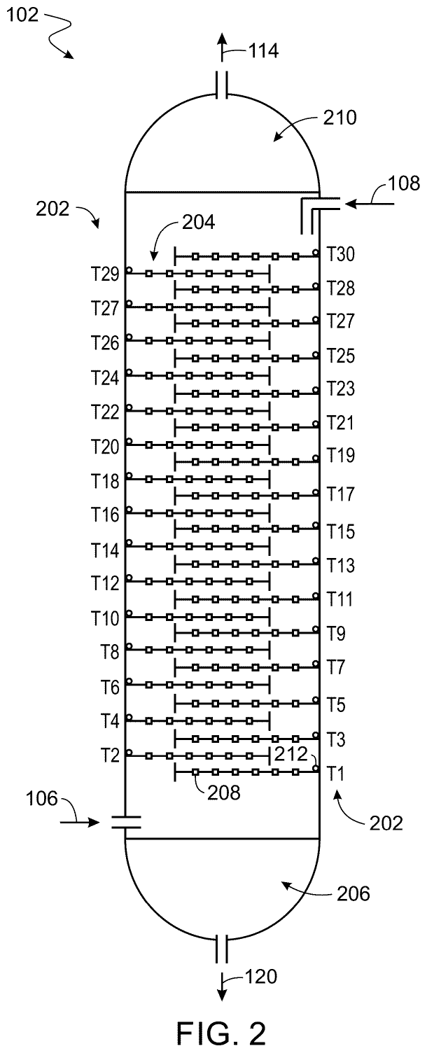

A temperature sensor 136 on a tray in the contactor 102, such as tray 6 counting up from the bottom, may be used to provide a temperature measurement to the controller. In some embodiments, as shown in the example of FIG. 2, each tray has a temperature sensor. In some embodiments, the tray temperature of each tray is calculated by the 1D-CNN. The temperature readings from trays can be used as a cross-check or as additional inputs to the 1D-CNN. As discussed below, the tray temperature is used to optimize the energy usage or sour gas flow rate, for example, as the highest tray temperature is higher in the column, this may indicate a higher probability of a breakthrough from the column.

The model in the controller 118 may be used to maximize the rate of the sour gas feed 106, for example, by adjusting a flow controller 138 on the sour gas feed 106. Further, the model in the controller 118 may be used to minimize the energy consumption, for example, by adjusting a flow control valve 140 on the which is used to adjust the flow of the lean solvent 108 fed to the contactor 102.

FIG. 2 is a cross sectional drawing of a contactor 102 used to absorb acid gases in a reactive absorption unit 100. Like numbered items are as described with respect to FIG. 1. As shown in FIG. 2, the contactor 102 in this example has 30 trays 202, labeled T1 to T30 from the bottom of the contactor 102.

In the contactor 102, a stream of lean solvent 108 is introduced near the top of the contactor 102, for example, above tray T30. The lean solvent 108 can be introduced at multiple points proximate to the top of the column, for example, above trays T30, T29, T28, and T27, in addition to other configurations. The lean solvent 108 flows across the tray T30, maintaining a liquid level on the tray T30 before flowing through a downcomer 204 to the next tray, T29. This continues through the contactor 102, as the solvent absorbs acid gases and drops from the bottom tray, T1, into the bottom head 206 of the contactor 102, prior to exiting the contactor 102 as a rich solvent 120.

The sour gas feed 106 is introduced to the contactor 102 proximate to the bottom, for example, below tray T1, and rises up through the contactor 102 through the trays 202. Each of the trays 202 has a number of bubble caps 208 to allow the sour gas feed 106 to pass through the tray and bubble through the solvent, allowing the acid gases in the sour gas feed 106 to be absorbed by the solvent, forming a sweetened gas stream 114, which exits the contactor 102 from the top head 210 of the vessel.

In this embodiment, each of the trays 202 has a temperature sensor 212. However, as described herein, the contactor 102 may have fewer temperature sensors or a single temperature sensor. As the absorption reaction is exothermic, the temperature profile, or temperature at each of the trays 202 is related to the amount of acid gas being absorbed at that tray. As the peak temperature moves to higher trays, the probability of a breakthrough, or contamination of the sweetened gas stream with acid gases, increases. As described herein, the tray temperatures can be modeled, for example, using a 1D-CNN.

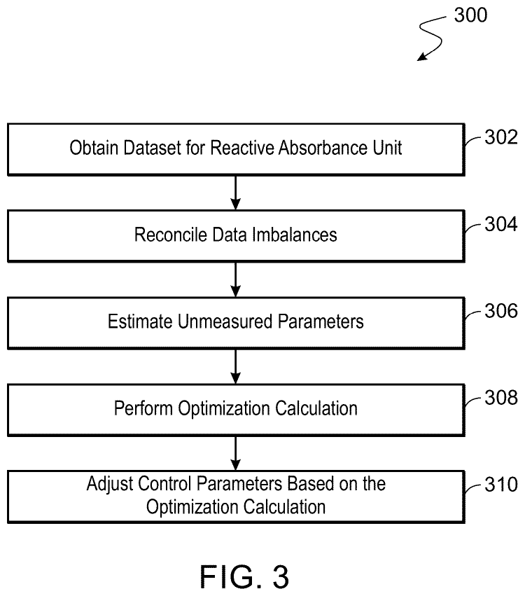

FIG. 3 is a process flow diagram of a method 300 for using a model to optimize operations of a reactive absorption unit. The method 300 begins at block 302 by obtaining latest dataset either from DCS or plant historian, based on the configuration. The dataset can be instantaneous or represent an average of a given period, such as an hour. The data is cleaned by removing outliers and adjusting the signals, for example, capping the values to the minimum and maximum limits.

At block 304, data reconciliation is performed. In this process, flowrates are mass balanced to ensure inlets equal outlets. For example, imbalances are distributed by distributing the imbalance based on each meter's perceived inaccuracy.

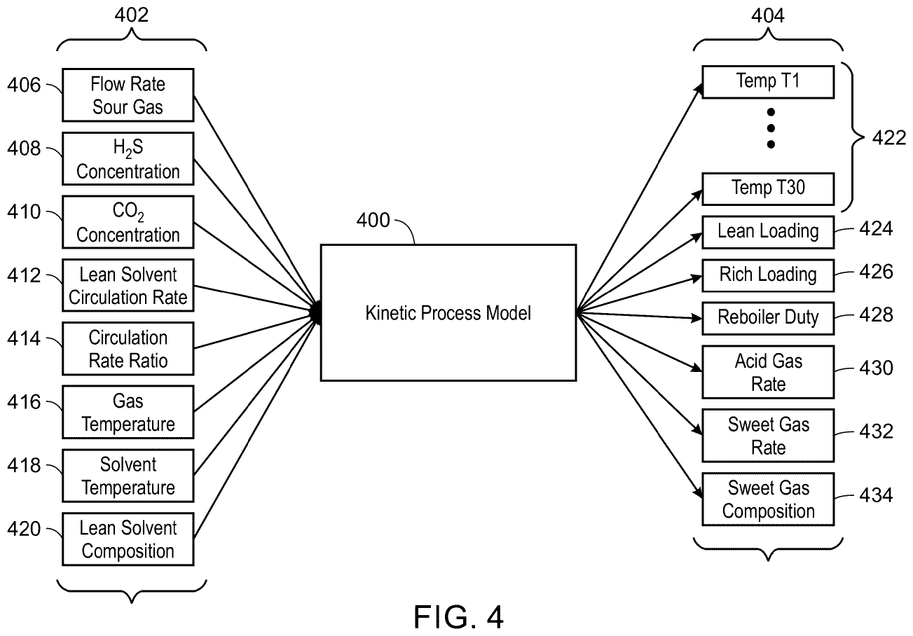

At block 306, parameter estimation is performed to estimate manipulated and disturbance variables that are not measured in real time. These values can include the composition of the feed composition and the composition of the aqueous solution when no analyzer is present. The estimation is performed using models that correlate the unmeasured variables with measured variables. The models can have the form of regression models, machine learning models, kinetic models or reinforcement learning models. This is discussed further with respect to FIG. 4.

At block 308, the optimization calculation is performed. The optimization algorithm solves two optimization problems, including a maximum throughput calculation and a minimum circulation rate calculation. The minimum circulation rate calculation minimizes energy consumption since the consumption is largely attributed to the circulation rate that controls pump power consumption and reboiler energy consumption at the stripper.

The optimization is performed at two instances, the parameter estimation-error minimization (if needed), and the two optimization problems. Differential evolution can be used to solve both optimization problems. The first optimization problem determines maximum throughput while meeting constraints relating to the temperature profile, lean loading, rich loading, and contactor flooding (if needed). The second optimization problem figures out the minimum circulation rate that can be used while meeting those same constraints. For example, to maximize throughput, the model is allowed to increase circulation rate up to the maximum that can be offered by the pumps. Similarly, to minimize circulation rate, the plant throughput is retained as the circulation rate is decreased.

To maximize the feed rate, the optimization solver is configured with an objective function relating to maximizing the feed gas rate. The optimization variables include the circulation rate and the reboiler duty. Constraints include the lean and rich minimum and maximum loading of the amine solution, the maximum rate of the circulation pumps, the maximum duty of the reboiler, the maximum cooling duty, the maximum flooding factor, and the impurities in the treated gas, such as H₂S and CO₂, among others. An important constraint is the overall temperature profile of the contactor. As described with respect to FIG. 2, the temperature profile indicates the likelihood of acid gas breakthrough in the top of the column. The temperature profile is constrained by specifying maximum temperature for the whole curve in addition to the highest tray at which the top temperature can occur, for example, specifying that the peak temperature cannot occur above T16 (FIG. 2).

To minimize the energy consumption, the optimization solver is configured with an objective function relating to minimizing the circulation rate of the aqueous solution, which leads to the minimum energy consumption. The optimization variables include the reboiler duty. Constraints include the aqueous solution lean and rich minimum and maximum loading, the circulation pumps minimum rate, the maximum flooding factor, the impurities in the treated gas, such as H₂S and CO₂, and any other operational limits that are considered key to the operation. An important constraint is also the overall temperature profile of the column, which indicates the likelihood of acid gas breakthrough in the top of the column. The temperature profile is constrained by specifying maximum temperature for the whole curve in addition to the highest tray at which the top temperature can occur, for example, the peak temperature cannot occur above tray #16 in trayed columns.

In the above description, controlled variables are described in terms of the input (manipulated and disturbance variables). This can be in the form of regression models, algebraic models or reinforcement learning models.

The optimization solver can be derivative-based mathematical solver, or a derivate free solver based on algorithms such as differential evolution, genetic algorithm, and simulated annealing. It can also be in the form of a reinforcement learning algorithm which is pre-trained on simulation data.

At block 310, the control parameters are adjusted based on the optimization calculation. In an open loop optimization, the results of both runs are displayed on a set of dashboards, and an operator may make the adjustments as desired. In a closed loop optimization, the user selects the desired operating mode for the unit, such as minimizing energy consumption at current gas rate or maximizing feed rate. The appropriate set of operating points are then passed accordingly to the plant control system.

The process is run automatically with a set frequency. Some steps may be performed at a different frequency than others. For instance, the parameter estimation step may be done less frequently since most estimated parameters are not expected to change in the short horizon.

FIG. 4 is a schematic drawing of a kinetic process model 400 used to estimate process values. In this example, the kinetic process model 400 was developed using the ProMax® software, available from Bryan Research & Engineering, LLC, which has specific capabilities for modeling the kinetics of acid gas amine absorption. However, the kinetic process model 400 can be constructed using any number of other commercially available process modeling packages, including Aspen Plus® available from Aspen Technology, Inc. The model is constructed by selecting process units in the modeling package that represent each type of process unit in the process, for example, as shown in FIG. 1. The process units are functionally coupled, and matching input and output parameters are selected for each coupled process unit. The final model provides an operational model of the process, for example, from initial inputs through final outputs.

The kinetic process model 400 was validated against plant data. The input parameters 402 included manipulated and disturbance variables, including such inputs as the flow rate for the sour gas feed 406, the H₂S content of the sour feed 408, the CO₂ content of the sour gas feed 410, the amine circulation rate 412, and the amine circulation rate ratio 414, which is the ratio between the flow rate of the sour gas feed 406 and the amine circulation rate 412. Further, the input parameters 402 may include the gas temperature 416, the amine temperature 418, and the lean amine composition 420, including the amine, methyldiethanolamine (MDEA) and piperazine (pz) concentration in the amine. A range for each of the variables can be selected based on plant performance, for example, by observing the plant actual operating limits for each of the variables. The lower margins and upper margins of each of the ranges may be adjusted to account for operations outside of the normal operating ranges. A sampling method is then used to generate a large set of values relating inputs to outputs. For example, in an embodiment, a Latin hypercube sampling method is used to generate 8000 multidimensional samples. Other sampling techniques can be used, including Sobol sampling, random sampling, and the like.

These samples are run in the model, and results, or output parameters 404, are collected for various parameters, including tray temperatures 422 in the contactor, the lean loading 424, the rich loading 426, among others. Other variables that can be included in the output parameters 404 include, for example, the reboiler duty 428, the acid gas flow rate 430, the sweet gas production rate 432, and the sweet gas composition 434.

The input parameters 402 and the output parameters 404 can be used as a training set to train machine learning models. For example, in some embodiments, a 1D-CNN is used to build a relationship between the input parameters 402 and the temperature profile of the tray temperatures 422. In some embodiments, a polynomial regression is used to build correlation of input parameters 402 with each of the lean loading 424 and rich loading 426. A parameter estimation algorithm was then set up, which solves its own optimization problem. Based on plant measurements, such as actual temperature profile, flowrates, and temperatures, the model estimates unmeasured output parameters 404, such as feed acidity, which would best explain the variations in the other parameters across multiple time stamps. For example, assuming feed acidity is constant over a week and that other parameters have been varying, resulting in varying outputs, the model would estimate the best value for acidity to minimize the difference between actual measurements of the input parameters 402 and the estimates of the output parameters 404 provided by the models we had developed. The machine learning model would try to find these best values for the unmeasured parameters.

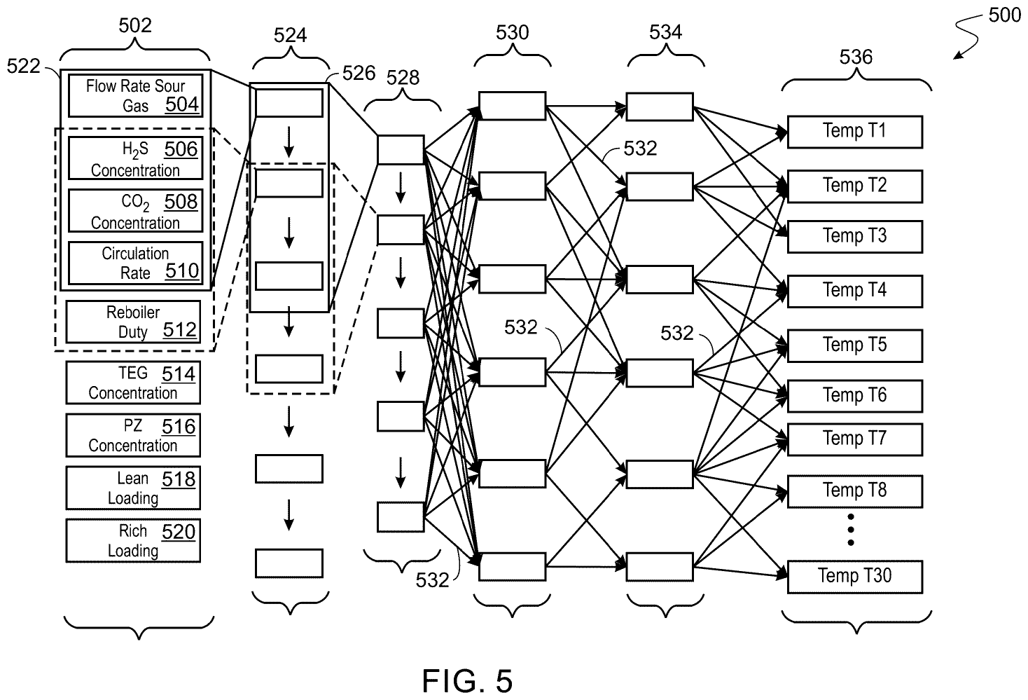

The estimated values can then be used in a further optimization process, either to provide missing values, if needed, or as a training set for a machine learning model that relates inputs to outputs. The machine learning model can vary in complexity and include a linear regression model, a polynomial regression model, a KNN (K-nearest neighbors algorithm), a convolutional neural network, or other machine learning models, such as a 1D-CNN. An example of this is discussed further with respect to FIG. 5.

FIG. 5 is a drawing of a one-dimensional convolutional neural network (1D-CNN) 500 that can be used to control the reactive absorption unit. It can be noted that this is an example of the inputs and outputs that may be used. However, as discussed above, some of the parameters may be calculated by other models, such as regression analysis or kinetic models, among others.

The input layer 502 for the 1D CNN is a sequence of values, wherein each element in the sequence corresponds to a feature. In the example shown in FIG. 5, the input layer 502 includes nodes for the parameters discussed above, including nodes for the flow rate of the sour gas 504, the concentration of the H₂S 506, the concentration of the CO₂ 508, the circulation rate 510 of the amine, and the reboiler duty 512. Other parameters that can be included are the concentration of triethylene glycol (TEG) 514 in the amine, the concentration of piperazine 516, the lean loading 518 of the amine, and the rich loading 520 of the amine. As used herein, the term loading indicates the concentration of the acid gases in the amine.

The parameters are not restricted to the ones shown in FIG. 5. Depending on the model inputs and the desired outputs from the 1D-CNN 500 fewer input parameters or more input parameters may be included. In some embodiments, input parameters handled by a different model, such as a kinetic model or a regression model, may not be included in the input parameters to the 1D-CNN 500.

The 1D CNN 500 is specifically used for processing one-dimensional sequences. A 1D filter, termed a kernel 522, is slid across the input sequence to extract relevant features. The kernel 522 computing a convolution operation for each position of a convolutional layer 524. The convolutional layer 524 of the 1D CNN 500 is a feature map of local patterns and features from the input sequence of the input layer 502.

After the creation of the convolutional layer 524, a down sampling operator 526 creates a pooling layer 528 by aggregating neighboring values in the convolutional layer 524. Common pooling methods include max pooling (selecting the maximum value in a window) or average pooling (taking the average of values in a window). An activation function, termed a ReLU, is used to introduce non-linearity to the model. In some embodiments, the ReLU function is f(x)=max(0,x), which sets all negative values to zero. The help the network learn complex relationships between features.

After convolution and pooling, a flatten layer 530 of nodes is created coupling the nodes of the pooling layer 528 to the nodes of the flatten layer 530 with hyper parameters 532 that are adjusted by training with data sets, for example, created as discussed with respect to FIG. 4. The flatten layer 530 is processed by the nodes of a fully connected layer 534, which process the flattened feature map. These layers 530 and 534 learn high-level representations and make predictions. The predictions correspond to the heads of the output layer 536.

In this embodiment, the outputs from the 1D CNN 500 are spatially dependent, for example, predicting trade temperatures to develop a contactor temperature profile. Each output head corresponds to a specific segment or location in the input sequence. By sharing lower-level features across all output heads, the model captures spatial dependencies.

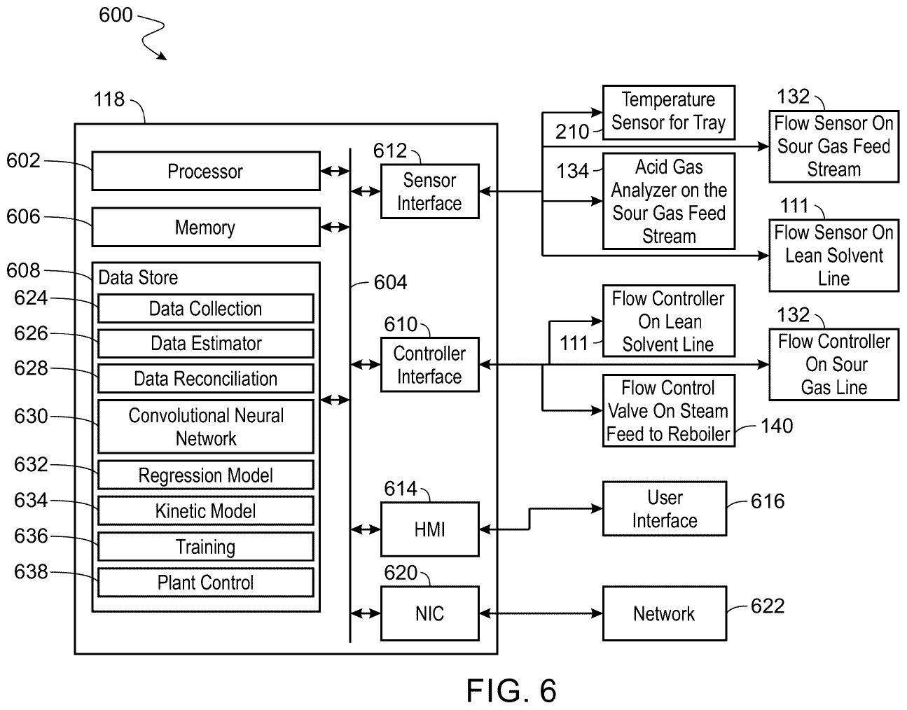

FIG. 6 is a block diagram of a system 600 that uses a controller 118 for optimizing operation of reactive absorption unit. Like numbered items are as described with respect to FIG. 1. As described herein, the controller 118 may be used to provide direct or advisory process control to maximize sour gas throughput or minimize energy demand, leading to more robust process control and higher efficiency.

In some embodiments, the controller 118 may be a separate unit mounted in the field or plant, such as a programmable logic controller (PLC), for example, as part of a supervisory control and data acquisition (SCADA) or Fieldbus network. In other embodiments, the controller 118 may reside in a distributed control system (DCS) installed in a central control center. In still other embodiments, the controller 118 may be a virtual controller running on a processor in a DCS, on a virtual processor in a cloud server, such as on a corporate network.

The controller 118 includes a processor 602. The processor 602 may be a microprocessor, a multi-core processor, a multithreaded processor, an ultra-low-voltage processor, an embedded processor, or a virtual processor. The processor 602 may be part of a system-on-a-chip (SoC) in which the processor 602 and other components are formed into a single integrated package. In various embodiments, the processor may include processors from Intel® Corporation of Santa Clara, California, from Advanced Micro Devices, Inc. (AMD) of Sunnyvale, California, or from ARM holdings, LTD., of Cambridge England. Any number of other processors from other suppliers may also be used, including proprietary processors used for DCS applications.

The processor 602 may communicate with other components of the controller 118 over a bus 604. The bus 604 may include any number of technologies, such as industry standard architecture (ISA), extended ISA (EISA), peripheral component interconnect (PCI), peripheral component interconnect extended (PCIx), PCI express (PCIe), or any number of other technologies. The bus 604 may be a proprietary bus, for example, used in an SoC based system. Other bus technologies may be used, in addition to, or instead of, the technologies above. For example, plant interface systems may include I2C buses, serial peripheral interface (SPI) buses, Fieldbus, and the like.

The bus 604 may couple the processor 602 to a memory 606. In some embodiments, such as in PLCs and other process control units, the memory 606 is integrated with a data store 608 used for long-term storage of programs and data. The memory 606 include any number of volatile and nonvolatile memory devices, such as volatile random-access memory (RAM), static random-access memory (SRAM), flash memory, and the like. In smaller devices, such as PLCs, the memory 606 may include registers associated with the processor itself. The data store 608 is used for the persistent storage of information, such as data, applications, operating systems, and so forth. The data store 608 may be a nonvolatile RAM, a solid-state disk drive, or a flash drive, among others. In some embodiments, the data store 608 will include a hard disk drive, such as a micro hard disk drive, a regular hard disk drive, or an array of hard disk drives, for example, associated with a DCS or a cloud server.

The bus 604 couples the controller 118 to a controller interface 610. The controller interface 610 may be an interface to a plant bus, such as a Fieldbus, an I2C bus, an SPI bus, and the like. The controller interface 610 couples the controller 118 to the flow controller of the flow sensor 111 on the lean solvent line to the contactor to assist in controlling the circulation rate. Although shown as part of the flow transmitter, it may be understood that the flow controller may be an independent code block in the controller, or a separate control block in a DCS. The controller interface 610 also couples the controller 118 to the flow controller of the flow sensor 132 on the sour gas feed 106, to control the sour gas feed rate to the contactor. The controller interface 610 can also couple the controller 118 to a flow control valve 140 on the steam feed to the reboiler to adjust the duty cycle. Although not shown, any number of other controls can be coupled to the controller 118 through the controller interface 610, such as a flow control valve on the sweetened gas stream 114 from the contactor 102 (FIG. 1).

A sensor interface 612 couples the controller 118 to a temperature sensor 212 (or a number of temperature sensors) for a tray in the contactor. The sensor interface 612 may be an interface to a plant bus, such as a Fieldbus, an I2C bus, an SPI bus, and the like. The sensor interface 612 also couples the controller 118 to the flow sensor 132 on the sour gas feed stream, the acid gas analyzer 134 on the sour gas feed stream (if present), and the flow sensor 111 on the lean solvent line. If present, the sensor interface 612 may also couple the controller 118 to an acid gas analyzer 116 on the sweet gas outlet stream from the contactor.

If the controller 118 is located in the field, a local human machine interface (HMI) 614 may be used to input control parameters. The local HMI 614 may be coupled to a user interface 616, including, for example, a display that includes a multiline LCD display, or a display screen, among others. The user interface 616 may also include a keypad for the entry of control parameters, such as the starting parameters for the flow of the lean solvent into the contactor. Generally, the controller 118 will either be part of a plant control system, such as a distributed control system (DCS), or coupled through a plant bus system to the plant control system. Thus, the control will be performed through the DCS control screens.

In some embodiments, the controller 118 is linked to the plant control system through a network interface controller (NIC) 620. The NIC 620 can be an Ethernet interface, a wireless network interface, or a plant bus interface, such as Fieldbus. The NIC 620 can also link the controller 118 to a network 622, such as a corporate network. In some embodiments, the controller 118 resides on the corporate network and communicates through the network 622 to the DCS for plant control. Further, the controller 118 can access a data historian on the DCS to obtain data for modeling and training purposes, as described herein.

The data store 608 includes blocks of stored instructions that, when executed, direct the processor 602 to implement the control functions for the contactor. The data store 608 includes a block 624 of instructions to direct the processor to collect data from the sensors through the sensor interface 612, or from the network 622, such as the data historian. The data collected can include the data used as the nodes of the input layer 502 for the 1D CNN 500, as described with respect to FIG. 5.

The data store 608 also includes a block 626 of instructions to direct the processor 602 to estimate values that are not collected. As described herein, this may be performed by running kinetic models based on the input parameters.

The data store 608 includes a block 628 of instructions to direct the processor 602 to perform data reconciliation on the data used. As described herein, that includes the balancing of inputs and outputs based on expected accuracy of various sensors, among others.

A block 630 of instructions may be included in the data store 608 to direct the processor 602 to perform calculations for a 1D-CNN, for example, as discussed with respect to FIG. 5. For example, the calculations may take the reconciled data in the input nodes and use the 1D-CNN to calculate the predicted values for the output nodes. The data store 608 includes a block 632 of instructions to direct the processor to use the regression model to estimate missing data points and calculate control outputs from data inputs. The data store 608 includes a block 634 of instructions to direct the processor to use a kinetic model to estimate output values from input parameters.

A block 636 of instructions may be included in the data store 608 to direct the processor 602 to train the 1D-CNN and the regression models, or other models used. These instructions may be used to adjust the hyper parameters 508 of the 1D CNN 500 by comparing values calculated for the nodes of the output layer 506 to values from training sets.

A block 638 of instructions may be included in the data store 608 to direct the processor 602 to provide plant control. This may be performed by having an operator select an operational mode, for example, to maximize sour gas feed rate or minimize energy consumption, then using the models to calculate adjustments and directly adjusting the valves and other plant systems through the sensor interface 612. The values and adjustments would then be displayed on an output screen of a plant control system. In some embodiments, this may be performed by displaying the output screen on the output screen of the plant control system and allowing the operators to make the adjustments to the and other control systems.

Any number of other blocks may be included in the data store 608 to implement other functions, including blocks of instructions to direct the processor to measure the acid gas concentrations in the sweetened gas stream 114 using an acid gas analyzer 116 on the outlet from the contactor 102.

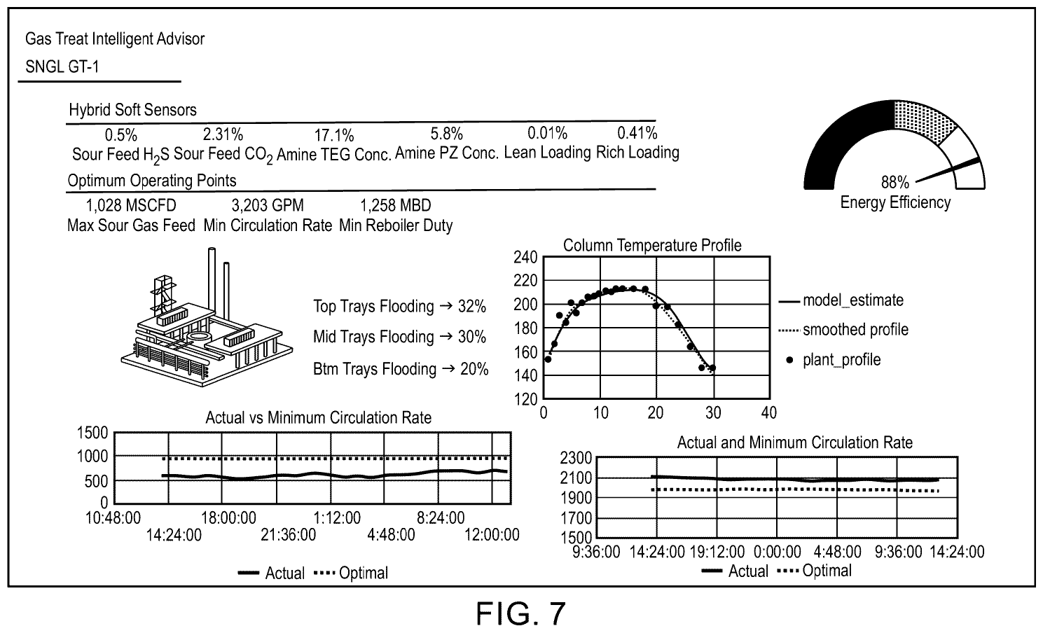

FIG. 7 is an example of an output screen that is used to control the reactive absorption unit, either through an open loop control or through a closed loop control. The output control screen displays the current measured and predicted values, the energy efficiency, and the temperature profile for the contactor, including both modeled and measured values, among other information.

### Embodiments

An embodiment described herein provides a method for estimating a parameter for a reactive absorbance unit. The method includes creating a kinetic model of an absorbance process, setting a range for each of a plurality of input parameters, based, at least in part, on operational data measured from the reactive absorbance unit. A sampling technique is used to generate a plurality of input vectors in the range of each of the plurality of input parameters. A plurality of output vectors is generated from the plurality of input vectors. A predictive model is trained with the plurality of output vectors and the plurality of input vectors. The parameter is estimated from the predictive model. The parameter is used in a control model for the reactive absorbance unit.

In an aspect, combinable with any other aspect, the method includes obtaining the operating data from a data historian in a plant.

In an aspect, combinable with any other aspect, the method includes obtaining the operating data from a distributed control system (DCS).

In an aspect, combinable with any other aspect, the method includes reconciling data imbalances by mass balancing flow rates for inlets and outlets. In an aspect, the method includes distributing the data imbalances based on an estimated accuracy of each meter.

In an aspect, the predictive model is a regression model developed from a mathematical correlation of outputs to inputs. In an aspect, the predictive model is a k-nearest neighbors (KNN) model.

In an aspect, combinable with any other aspect, the sampling technique is a Sobol sampling method.

In an aspect, combinable with any other aspect, the sampling technique is random sampling method.

In an aspect, combinable with any other aspect, the sampling technique is a Latin hypercube sampling method.

In an aspect, combinable with any other aspect, the plurality of input parameters includes a flow rate for a sour gas feed, an amine circulation rate, or an amine circulation rate ratio, or any combinations thereof.

In an aspect, combinable with any other aspect, the plurality of input parameters includes a sour gas temperature, a lean amine temperature, or a lean amine composition, or any combinations thereof.

In an aspect, combinable with any other aspect, the plurality of output vectors includes tray temperatures in a contactor, a lean amine loading, or a rich amine loading, or any combinations thereof.

In an aspect, combinable with any other aspect, the plurality of output vectors includes a reboiler duty, an acid gas flow rate, a sweet gas production rate, or a sweet gas composition, or any combinations thereof.

Another embodiment described herein provides a reactive absorbance unit. The reactive absorbance units includes a contactor. A feed gas line is coupled to the contactor proximate to the bottom of the contactor, wherein the feed gas line includes a sour gas feed. A sweetened gas line from the contactor is coupled proximate to a top of the contactor, wherein the sweetened gas line includes a sweetened gas. A lean amine line to the contactor is coupled proximate to the top of the contactor, wherein the lean amine line includes a lean amine solvent. A rich amine line from the contactor is coupled proximate to a bottom of the contactor, wherein the rich amine line includes an amine solvent with absorbed acid gases. The reactive absorption's unit includes a stripper, wherein the rich amine line is coupled to the stripper, and wherein the lean amine line exits the stripper after removal of acid gases. The reactive absorbance unit further includes a control system. The control system includes a processor and a data store. The data store includes instructions configured to direct the processor to obtain a data set for reactive absorbance unit, reconcile data imbalances in the data set, generate a plurality of input parameters for a kinetic model, run the plurality of input parameters in the kinetic model, and to generate a plurality of output parameters from the kinetic model. The data store also includes instructions configured to direct the processor to create a machine learning model relating input parameters to output parameters, and run new input parameters in the machine learning model to estimate an unmeasured parameter, creating an estimated parameter. The data store also includes instructions configured to direct the processor to perform a control calculation using the estimated parameter.

In an aspect, combinable with any other aspect, the plurality of input parameters includes a plurality of multidimensional vectors sampled across a range of each of the plurality of input parameters.

In an aspect, combinable with any other aspect, the machine learning model includes a polynomial equation generated from a regression analysis of the plurality of input parameters with the corresponding one of the plurality of output parameters.

In an aspect, combinable with any other aspect, the machine learning model includes a one-dimensional, convolutional neural network trained using the plurality of input parameters with the corresponding plurality of output parameters.

In an aspect, combinable with any other aspect, the control system includes a network interface card, and wherein the data store includes instructions configured to direct the processor to access a data historian to obtain the data set.

In an aspect, combinable with any other aspect, the control system includes a network interface card, and wherein the data store includes instructions to direct the processor to display the estimated parameter on a display screen.

Other implementations are also within the scope of the following claims.

What is claimed is:

1. A method for estimating a parameter for a reactive absorbance unit, comprising:
creating a kinetic model of an absorbance process;
setting a range for each of a plurality of input parameters, based, at least in part, on operational data measured from the reactive absorbance unit;
using a sampling technique to generate a plurality of input vectors in the range of each of the plurality of input parameters;
generating a plurality of output vectors from the plurality of input vectors;
training a predictive model with the plurality of output vectors and the plurality of input vectors;
estimating the parameter from the predictive model; and
using the parameter in a control model for the reactive absorbance unit.

2. The method of claim 1, comprising obtaining the operating data from a data historian in a plant.

3. The method of claim 1, comprising obtaining the operating data from a distributed control system (DCS).

4. The method of claim 1, comprising reconciling data imbalances by mass balancing flow rates for inlets and outlets.

5. The method of claim 4, comprising distributing the data imbalances based on an estimated accuracy of each meter.

6. The method of claim 1, wherein the predictive model is a regression model developed from a mathematical correlation of outputs to inputs.

7. The method of claim 1, wherein the predictive model is a k-nearest neighbors (KNN) model.

8. The method of claim 1, wherein the sampling technique is a Sobol sampling method.

9. The method of claim 1, wherein the sampling technique is random sampling method.

10. The method of claim 1, wherein the sampling technique is a Latin hypercube sampling method.

11. The method of claim 1, wherein the plurality of input parameters comprises a flow rate for a sour gas feed, an amine circulation rate, or an amine circulation rate ratio, or any combinations thereof.

12. The method of claim 1, wherein the plurality of input parameters comprises a sour gas temperature, a lean amine temperature, or a lean amine composition, or any combinations thereof.

13. The method of claim 1, wherein the plurality of output vectors comprises tray temperatures in a contactor, a lean amine loading, or a rich amine loading, or any combinations thereof.

14. The method of claim 1, wherein the plurality of output vectors comprises a reboiler duty, an acid gas flow rate, a sweet gas production rate, or a sweet gas composition, or any combinations thereof.

15. A reactive absorbance unit, comprising:
a contactor;
a feed gas line to the contactor coupled proximate to the bottom of the contactor, wherein the feed gas line comprises a sour gas feed;
a sweetened gas line from the contactor, coupled proximate to a top of the contactor, wherein the sweetened gas line comprises a sweetened gas;
a lean amine line to the contactor coupled proximate to the top of the contactor, wherein the lean amine line comprises a lean amine solvent;
a rich amine line from the contactor coupled proximate to a bottom of the contactor, wherein the rich amine line comprises an amine solvent with absorbed acid gases;
a stripper, wherein the rich amine line is coupled to the stripper, and wherein the lean amine line exits the stripper after removal of acid gases; and
a control system, comprising:
a processor; and
a data store, wherein the data store comprises instructions configured to direct the processor to:
obtain a data set for reactive absorbance unit;
reconcile data imbalances in the data set;
generate a plurality of input parameters for a kinetic model;
run the plurality of input parameters in the kinetic model, to generate a plurality of output parameters from the kinetic model;
create a machine learning model relating input parameters to output parameters; and
run new input parameters in the machine learning model to estimate an unmeasured parameter, creating an estimated parameter; and
perform a control calculation using the estimated parameter.

16. The reactive absorbance unit of claim 15, wherein the plurality of input parameters comprises a plurality of multidimensional vectors sampled across a range of each of the plurality of input parameters.

17. The reactive absorbance unit of claim 15, wherein the machine learning model comprises a polynomial equation generated from a regression analysis of the plurality of input parameters with the corresponding one of the plurality of output parameters.

18. The reactive absorbance unit of claim 15, wherein the machine learning model comprises a one-dimensional, convolutional neural network trained using the plurality of input parameters with the corresponding plurality of output parameters.

19. The reactive absorbance unit of claim 15, wherein the control system comprises a network interface card, and wherein the data store comprises instructions configured to direct the processor to access a data historian to obtain the data set.

20. The reactive absorbance unit of claim 15, wherein the control system comprises a network interface card, and wherein the data store comprises instructions to direct the processor to display the estimated parameter on a display screen.
